(19) United States
(12) Patent Application Publication
(10) Pub. No.: US 2009/0170153 A1
(43) Pub. Date: Jul. 2, 2009

(54) EFFECT OF RADIATION ON CELLULASE ENZYMES

(75) Inventor: Earnest Stuart, East Lansing, MI (US)

Correspondence Address: WARNER NORCROSS & JUDD LLP, 900 FIFTH THIRD CENTER, 111 LYON STREET, N.W., GRAND RAPIDS, MI, 49503-2487, US

(73) Assignee: RA ENERGY, East Lansing, MI (US)

(21) Appl. No.: 11/995,154

(22) PCT Filed: Jul. 11, 2005
(86) PCT No.: PCT/US05/24166

§ 371 Date: Jul. 23, 2008

Related U.S. Application Data
(60) Provisional application No. 60/586,611, filed on Jul. 9, 2004.

(51) Int. Cl. C12P 21/00; C12P 7/00; C12P 19/04; C12N 15/01; C12N 9/00

(52) U.S. Cl. 435/68.1; 435/132; 435/101; 435/446; 435/183

(57) ABSTRACT

A method for recycling cellulase enzymes. Also provided is a method for producing fermentable carbohydrates, plant leaf protein, and lignin, by adding a cellulase enzyme complex expressed from and on irradiated cellulase complex-producing organisms with sufficient radiation to kill biological activity without destroying all cellulase enzyme complex activity to biomass. The fermentable carbohydrates produced by the method. Also provided are irradiated cellulase-producing organisms for use in converting biomass to fermentable sugars, plant leaf protein, and lignin. A method for producing cellulase enzymes for glucose and other sugar production and protein and lignin extraction by irradiating cellulase-producing organisms, thereby producing the cellulase enzymes is also provided. A system for producing fermentable carbohydrates, plant protein, and lignin, said system comprising irradiated cellulase-producing organisms and biomass is provided.

### BACKGROUND OF THE INVENTION

1. Technical Field

Generally, the present invention relates to a method for producing and recycling enzymes used for refining biomass. More specifically, the present invention relates to a method of applying radiation to produce enzymes and to re-use the enzymes one or more times in refining biomass into fermentable sugars and downstream products, including, but not limited to, ethanol.

2. Description of the Related Art

The market for glucose from biomass, despite the failure so far of anyone to break through to an economical biomass refining method, is in the tens of billions of dollars per annum, and may ultimately rise to as high as $100-200 billion per annum worldwide as oil supplies dwindle. With oil prices rising and threatening to rise even higher, the demand for an alternative to gasoline is growing.

An individual cell of biomass is a sack with walls designed by nature which allow water into the cellular cavity, but which filters out proteins such as cellulase that would destroy the cell from within by dissolving its components, if access were gained. Once a breach of the cell wall occurs, there is no defense against hydrololytic attack by the cellulase family of enzymes. During a successful invasion of the cells by cellulase enzymes, the rate of cellulose and hemicellulose hydrolysis into fermentable sugars is extremely fast and highly complete with relatively low enzyme weight ratios to the biomass. Recent advances in estimated cellulase costs for ethanol production (By companies such as Genencor and Novozymes) have reduced the reported costs from $5/gallon of ethanol for the enzymes to $0.50/gallon of ethanol, and press releases by Novozymes indicates a near-term anticipated cost of under $0.30/gallon for the enzymes. Combined with a cost-effective treatment of biomass with lower cost enzymes, ethanol production cost estimates should rapidly drop into practical and commercial levels. One way to lower the production cost of ethanol further is by recycling cellulase enzymes used to extract glucose from biomass by hydrolysis. To date, no industrial method has been devised to recycle cellulase enzymes. Various methods to dissolve or treat biomass and/or prepare for enzymatic hydrolysis have been devised and tested over the last 30 years. Such methods include: concentrated acid, dilute acid/high temperature, steam, moderate temperature/neutral pH, dry grinding, strong alkali agents and peroxide, liquid anhydrous ammonia, high water ratios with lime, conically-shaped rotating rotor-stator tools, a lab sonicator applied to office paper are some of the methods which have been tested over the last 30 years or longer. Some use of a liquid stream, high-shear, and cavitating devices such as the Supraton disclosed in prior art have been used to treat biomass to affect a high degree of reactivity to enzymes (U.S. Pat. Nos. 5,370,999 and 5,498,766 to Stuart et al). While achieving success at producing high percentage hydrolysis of all biomass glucose using low loadings of cellulase enzymes applied to treated grass, the level of energy and capital required in this application without employing other parameter changes required to achieve such results, was not found to be economical. However, conducted on a more limited scale, the method is one practical shearing method, which is necessary for particle size reduction in preparation for new and novel methods. There is one promising method using a combination of cavitation, pH, and other parameters being developed which can cost-effectively dissolve the hemicellulose and the cellulose into monomer, fermentable sugars to a high percentage. All methods except concentrated acid employ cellulase enzymes in producing glucose from biomass. Presently, according to companies producing cellulase, as well as the National Renewable Energy Laboratories and others, the best guess cost of cellulase in biomass refining to ethanol is approximately $0.50/gallon (U.S.). This cost is presently prohibitive to economical ethanol production at the current price for oil and gasoline.

Additionally, methods have been devised using low-dose radiation for protecting sensitive components of blood and other high-value materials from contamination. But this method has not been applied to recycling cellulase enzymes and does not use higher radiation doses. Another method has been devised utilizing polymers that dissolve under specific conditions and can be repeatedly precipitated to attract cellulase, then release cellulase enzymes in the dissolving side of the process. Much similar research and testing has been done to recover enzymes from the liquid portion of a hydrolysis broth, but since most cellulase is attached to the solids, this method has not proven effective. No known commercial methods have been devised for recycling cellulase enzymes in a biomass refining process where the product is low in unit value, but high in market volume. A method employing enzymes from the cellulase family would benefit economically from a method to recycle cellulase enzymes within a biomass refining process.

### SUMMARY OF THE INVENTION

According to the present invention, there is provided a method for producing fermentable carbohydrates, plant leaf protein, and lignin, by adding a cellulase enzyme complex expressed from and on irradiated cellulase-complex-producing organisms, and/or post-hydrolysis biomass containing active cellulase enzymes having been treated with sufficient radiation to kill biological activity without destroying all cellulase enzyme complex activity, to biomass for hydrolysis, and the fermentable carbohydrate produced by the method. Also provided are irradiated, recyclable active cellulase for use in converting biomass to fermentable sugars, plant leaf protein, and lignin, and irradiated active cellulase associated with host organisms in the production of cellulase. A method for producing cellulase enzymes for glucose and other sugar production and protein and lignin extraction by irradiating cellulase-producing organisms, thereby producing the cellulase enzymes, and a method for recycling the cellulase is also provided. A system for producing fermentable carbohydrates, plant protein, and lignin, the system includes irradiated cellulase-producing organisms and biomass is provided.

### BRIEF DESCRIPTION OF THE DRAWINGS

Other advantages of the present invention are readily appreciated as the same becomes better understood by reference to the following detailed description, when considered in connection with the accompanying drawings wherein:

FIG. 1 is a graph showing biomass components, including glucose, xylose, protein, lignin, trace sugars, and lignin;

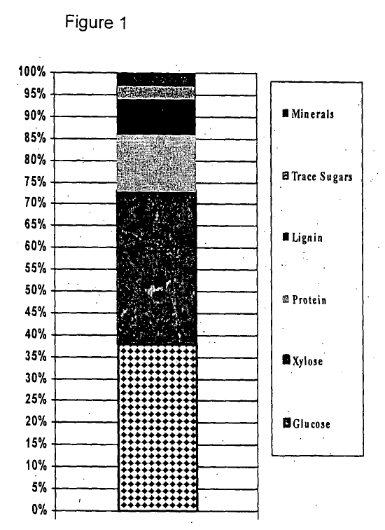

FIG. 2 is a photograph of biomass disrupted using cavitation; the particle sizes shown are typically well above 5 micron, and range up to several millimeters in length, as photographed utilizing an electron microscope at Texas A&M University;

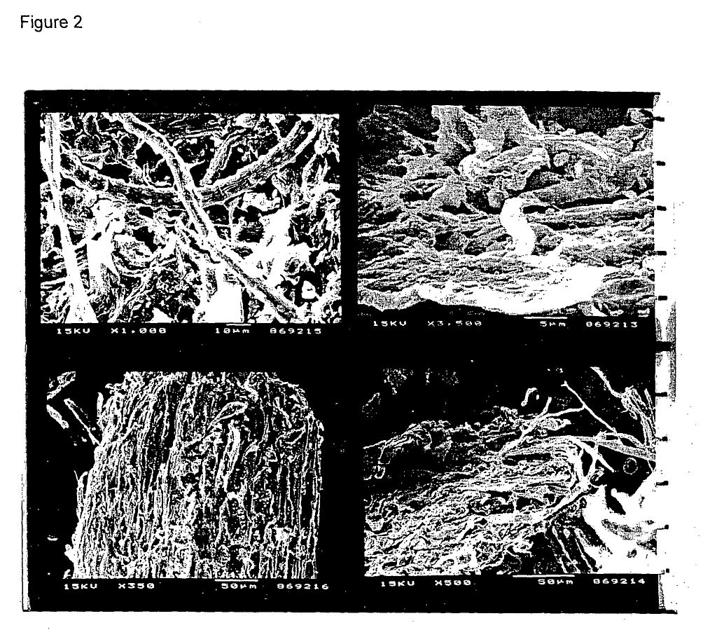

FIG. 3 is a graph showing the effect of irradiation on cellulase enzymes activity that have been treated in the dry form with various levels of cellulase; and

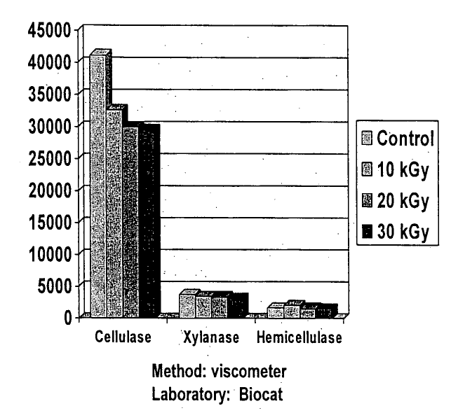

FIG. 4 is a bar graph depicting the effects of irradiation on cellulase enzyme activity that have been treated in the wet form.

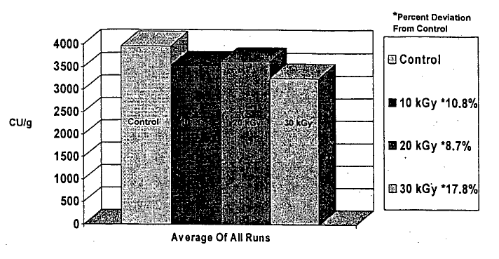

### DETAILED DESCRIPTION OF THE INVENTION

Generally, the present invention provides a method for producing fermentable carbohydrates, plant leaf protein, and lignin utilizing cellulase enzymes for producing organic chemicals, including, but not limited to, ethanol. More specifically, the present invention provides a method for producing and recycling enzymes having been treated with sufficient radiation to reduce or kill biological activity while preserving practical levels of cellulolytic activity and without destroying all cellulase enzyme complex activity, added to biomass for hydrolysis

As used herein, the term “biomass” includes any organic matter (whole, fractions thereof, and/or any components thereof) available on a renewable basis, such as dedicated energy crops and trees, agricultural food and feed crops, agricultural crop wastes and residues, wood wastes and residues, aquatic plants, animal wastes, municipal wastes, and other waste materials. Such biomass materials serve as raw materials for the process of the present invention. Additionally raw materials include, but are not limited to, cellulose-containing materials such as corn-fiber, hay, sugar cane bagasse, starch-containing cellulosic material such as grain, crop residues, newsprint, paper, sewage sludge, aquatic plants, sawdust, yard wastes, biomass, components thereof, fractions thereof, and any other raw materials or biomass materials known to those of skill in the art. Preferred is non-woody biomass generally having a lignin content of up to 18 percent, which includes woody biomass which has been treated in such a way as to remove some or most of its lignin. Lignocellulose-containing fiber, herein referred to as “biomass”, can potentially be refined into sugars, protein, and lignin, and chemicals for gasification into methane or hydrogen production.

More specifically, the term “biomass” as used herein can include any carbon-based materials. Biomass includes, but is not limited to, trees, grass, straw, grain husks, stalks, stems, leaves, aquatic plants such as water hyacinths, duckweed, paper, wood, etc. Preferably, the material of greatest volume that is used is a grass. Examples of grasses include, but are not limited to, (Axonopus affinis and Axonopus compressus), centipede grass (Eremochloa ophiuroides, buffalo grass (Buchloe dactyloides), hurricane grass also called Seymour grass (Bothriochloa Pertusa), and seashore paspalum (Paspalum vaginatum) additionally, other grasses that can be used include Poa, P. schistacea, P. xenica, Deyeuxia lacustris, Dichelachne lautumia, Brachiaria Mutica, acorus, andropogon, carex, festuca, glyceria, molina, panicum, phalaris, spartina, sporobolus, and miscanthus.

Biomass contains varying percentages of the following valuable components: glucose, xylose, trace carbohydrates, protein, minerals, and the glue, which bind these altogether, lignin. The percentage of lignin and other components are associated with the type of plant within general plant types such as grass, straw, aquatic, and wood, respectively. For example, wood tends to have lignin content of in excess of 22% in young trees, to in excess of 30% in mature trees. These levels of lignin allow trees to grow tall and hard but remain flexible enough to endure wind without falling over. Grass, on the other hand, is very soft and can contain lignin in an amount of less than 3% to as high as 11%. Environment and nutrient conditions can cause a wide range of component percentages in biomass.

The biomass used in conjunction with the method of the present invention can be pretreated. One example of such pretreatment is cavitation, which mechanically reduces particle size while creating extensive internal fissures through which cellulase enzymes can pass easily to access deeper biomass components not currently accessible. Other methods known to those of skill in the art for pretreatment of biomass can be utilizes without departing from the spirit of the present invention. Alternative pretreatment methods known to those of skill in the art can also be used without departing from the spirit of the present invention.

Cellulase enzymes consist of a so called “cocktail” of cellulose dissolving enzymes, which are proteins of multiple shapes and bio-electrical-chemical formulae, performing different functions in breaking down visible pieces of biomass into monomer sugars (1.5 nanometers; by comparison a DNA helix is 2 nanometers). There are cellulases that attack the gross structure of biomass and others that specialize in breaking down strings of complex carbohydrate structures, and others that break down two, three or more sugar molecule structures as precursors to monomer sugars. Therefore, throughout the present application whenever the term “cellulase enzymes” is used, the definition is intended to encompass the multi-functional cocktail of enzymes described above.

The present invention applies irradiation as quickly as commonly available, related equipment allows at a rate suitable for minimal damage to the cellulolytic activity of the enzymes, while destroying commercially practical levels of biological activity in organism which can consume the valuable sugars to be used for ethanol and other chemical fermentation. In the present invention, it is not necessary to preserve all cellulolytic activity to achieve a commercial value. In the present invention, irradiation can be applied in shorter timeframes than prior art, since recently-generated data has shown that even high levels of rapidly-applied irradiation (80 kilogray [kGy]) does not damage cellulase enzymes to the degree that they are not technically or economically viable. Few organisms require 80 kGy to kill biological activity in a single dose. Typically, approximately 25 kGy is sufficient for the method of the present invention for refining biomass. However, in a preferred embodiment, irradiation is first applied at a dosage sufficient for destroying or reducing biological activity in a given substrate, without destroying all or major levels of cellulolytic activities, followed by the repeated application of a subsequent dose or doses within such a process, which can total, with all doses, to as much as 80 kGy, or more. This offers the opportunity to effectively “recycle” the cellulase by applying the irradiated cellulases repeatedly to additional treated biomass between radiation doses, thus lowering the effective final cost of cellulase within a given process for hydrolyzing biomass.

A recent patent teaches the utilization of low-dose cobalt irradiation to sterilize various types of feedstock, which claims to eliminate limitations employed in prior art methods in research and commercial applications. A method for sterilizing products to inactivate biological contaminants such as viruses, bacteria, yeasts, molds, mycoplasmas and parasites is disclosed. The method involves irradiating the product at a low dose rate from about 0.1 kGy/hr to about 3.0 kGy/hr for a period of time sufficient to sterilize the product. The method does not destroy sensitive materials such as blood and blood components. Further, the method does not require pre-treatment of the product such as freezing, filtration or the addition of chemical sensitizers.

The present invention is distinct from prior art teachings in that there is no need to limit utilization radiation to low dosages described above administered over longer time to protect the cellulase enzymes which are surprisingly far more resistant to irradiation damage than biological materials previously referenced in the literature, particularly protein, while resident organisms can be mostly or completely destroyed. Historically, dosage to destroy living organisms, including bacteria, fungi and viruses, has been believed by those skilled in the art to be 25 kGy. More recent studies indicate that such results vary between contaminates and/or substrates in which they may be in residence, for a number of suggested reasons. That is to say, the specific dose of irradiation to kill an organism can be higher or lower than 25 kGy, depending on many factors, including density of substrate. The method of the present invention overcomes such variables by having demonstrated the ability to preserve cellulolytic activity even at extreme doses of irradiation delivered in very short timeframes and can therefore be tailored to a particular substrate and contaminant by applying irradiation rapidly and at a very wide range of doses.

It was previously thought that high dose irradiation, especially over a short timeframe, destroys all, or virtually all desirable active value in feedstock components, particularly protein: “Gamma irradiation is effective in destroying viruses and bacteria when given in high total doses. (Keathly, J. D. Et al.; Is There Life after Irradiation? Part 2; BioPharm July-August, 1993, and Leitman, Susan F.; Use of Blood Cell Irradiation in the Prevention of Post Transfusion Graft-vs-Host Disease; Transfusion Science 10:219-239, 1989). However, the published literature in this area teaches that gamma irradiation can be damaging to radiation sensitive products such as blood. In particular, it has been shown that high radiation doses are injurious to red cells, platelets and granulocytes (Leitman, ibid). Van Duzer, in U.S. Pat. No. 4,620,908 discloses that the product must be frozen prior to irradiation in order to maintain the viability of a protein product. Van Duzer concludes that: “If the gamma irradiation were applied while the protein material was at, for example, ambient temperature, the material would be also completely destroyed, that is the activity of the material would be rendered so low as to be virtually ineffective.” In contradistinction to the prior art teachings, the method of the present invention can be practiced at ambient temperatures.

The most recent patent issued on the application of irradiation to feedstock containing sensitive materials claims to solve the problem of damaging sensitive components while destroying biological activity by applying low doses of cobalt irradiation, discounting any method using high doses, while identifying the only known effective method so far: “In view of the above, there is a need to provide a method of sterilizing products that is effective in removing biological contaminants while at the same time having no adverse effect on the product. The present invention has shown that if the irradiation is delivered at a low dose rate, then sterilization can be achieved without harming the product. No prior references have taught or suggested that applying gamma irradiation at a low dose rate can overcome the problems admitted in the prior references.” The level of irradiation application is laid out: “Accordingly, the present invention provides a method for sterilizing a product comprising irradiating the product with gamma irradiation at a rate from about 0.1 kGy/hr. to about 3.0 kGy/hr. for a period of time sufficient to sterilize the product. The rate of irradiation can be specifically from about 0.25 kGy/hr. to about 2.0 kGy/hr., more specifically from about 0.5 kGy/hr. to about 1.5 kGy/hr. and even more specifically from about 0.5 kGy/hr. to about 1.0 kGy/hr. The length of time of irradiation or the total dose of irradiation delivered depends on the bioburden of the product, the nature of the contaminant and the nature of the product.”

In addition to strictly employing low doses of irradiation, the most recent related patent references timeframes significantly longer than the present invention: “Higher doses of irradiation are required to inactivate viruses as compared to bacteria. For example, using the dose rates of the present invention, one can use an irradiation time of greater than 10 hours to eliminate viral contamination in contrast to an irradiation time of only 45 minutes to remove bacterial contamination. The process according to the present invention may be carried out at ambient temperature and does not require the heating, freezing, filtration or chemical treatment of the product before the process is carried out. This offers another significant advantage of the method of the present invention as it avoids some of the extra treatment steps of the prior art processes.”

The literature indicates that dilution of substrate is often desirable, and in some cases, necessary to avoid degradation. In the present invention, a higher density of the irradiated cellulase protein has not contributed significantly to degradation of activity. The most recent related patent states: “Certain products, such as blood, can be diluted prior to irradiation. Diluting the product can serve to reduce degradation of the product during irradiation. The choice of diluent depends on the nature of the product to be irradiated. For example, when irradiating blood cells one would choose a physiologically acceptable diluent such as citrate phosphate dextrose. . . . Further, extremely sensitive products, such as blood, are preferably diluted in a physiologically acceptable diluent prior to irradiation.”

The most recently-issued related patent teaches that, prior to its issuance, there were no prior or existing methods for destroying bacteria, fungi and viruses, while preserving usefulness of valuable selected components: “The efficacy of the method of the present invention is contrary to what others skilled in this area have observed or predicted. (U.S. Pat. No. 4,620,908 and Susan Leitman, ibid). The method provides a method of irradiating products that is not harmful to the product itself. In particular, the method of the present invention can effectively sterilize a product as fragile as blood without destroying the viability of the cells contained therein. Consequently the method of the present invention offers a significant technical and scientific advancement to the sterilization field.”

The present invention offers significant advantages over prior art as it relates to preserving the cellulolytic activity of cellulase and other enzymes utilized in biomass refining, while destroying most or all other biological activity in the biomass substrate.

Referring specifically to the method of the present invention, the method of the present invention includes a step of irradiating and destroying, or partly destroying, the life of living fungi and/or bacteria, while simultaneously causing minimal damage to cellulase enzymes within the cellulase complex associated with, and produced by the living organisms up to the point of their engineered death by means of irradiation. The irradiated fungi and/or bacteria are introduced into a vessel containing biomass, which can optionally be irradiated to destroy living organisms and/or the spores contained in the biomass to varying degrees, ideally completely, which when mixed together, hydrolyzes the biomass into sugars that can be converted to organic chemicals, including ethanol, by way of fermentation. The irradiation of the fungi and/or bacteria does not destroy all cellulolytic activity of all the cellulase enzymes that are found within, on, or near the host fungi or bacteria. Thus, the fungi and/or bacteria can still hydrolyze the biomass into sugars including glucose, xylose, and other components in the presence of the dead host organisms, without competition for the sugars by the organisms. Freshly-produced, irradiated, and/or non-irradiated cellulase can be added to biomass for hydrolysis purposes at a point in the process before contamination can occur because of germinating spores, after which time the cellulase-containing biomass is irradiated to destroy biological activity. Alternatively, cellulase production methods can contain a step to irradiate the cellulase and/or the host organism prior to adding the cellulase to biomass for hydrolysis. Irradiation can be applied prior to the onset of contamination in order to extend hydrolysis times without further contamination.

Carbohydrates hydrolyzed from biomass using irradiated cellulases can then be used to form organic chemicals, including ethanol. Use of the irradiated enzymes enables a lower cost of production of ethanol because the enzymes are not ultra purified, a method commonly used by companies that manufacture cellulase enzymes to protect proprietary organisms. Lower-cost tank and stirring designs and recycling of cellulase enzymes can be employed to further reduce production costs.

Cellulase-producing organisms, cellulase and/or biomass irradiation is the process of exposing cellulase-producing organisms, cellulose, and/or biomass to controlled levels of a particular form of electromagnetic energy known as ionizing radiation. This term is used to describe these rays of energy because they cause whatever material they contact to produce electrically charged particles called ions.

Ionizing radiation is a part of the spectrum of electromagnetic energy that includes a type of energy similar to radio and television waves, microwaves and infrared radiation. However, the higher frequency and hence higher amount of energy produced by ionizing radiation allows it to penetrate deeply into cellulase-producing organisms, enzymes, and/or biomass, thereby killing microorganisms without significantly raising the enzyme or biomass temperature.

Irradiation disrupts the DNA strands in pathogenic microorganisms, such as bacteria, yeasts and molds, thereby either destroying the organism or preventing its reproduction. Scientists often compare the process to thermal pasteurization of milk. Three types of ionizing radiation can be used for irradiating cellulase-producing organisms, cellulase and biomass: gamma rays, high-energy electrons, which are sometimes referred to as electron beams (or e-beams), and X-rays. Until recently, gamma rays have been the exclusive source of food irradiation in the United States and elsewhere. While these three types of ionizing radiation have the same effects on food, there are some differences in how they function.

Gamma ray technology uses the radiation emanating from a radioactive substance, typically Cobalt 60, which is a radioactive isotope of the element cobalt. Cobalt 60 emanates high-energy photons, called gamma rays, which can penetrate biomass to a depth of several feet.

Electron beam and X-ray irradiators, irradiation facilities, are operated by electricity and do not use radioactive isotopes. The newest technology is X-ray irradiation. Examples of such machines are known to those of skill in the art.

Contrary to common expert belief, it was found that the use of irradiation does not totally affect the viability of cellulase enzymes, and indeed, at most levels of irradiation from 10-69 kGy, does not diminish cellulolytic activity more than 20%. The amount of irradiation used in the method of the present invention is directly linked to what is sufficient to destroy most or all bacterial and fungal life in a given cellulase host or grass substrate, without negatively affecting all the cellulase enzyme activity to a point where its value is too low for commercial purposes. It is generally believed that 25 kGy can kill most organisms by disrupting its DNA. The irradiation enables the cellulase enzymes of the fungi and/or bacteria to function in the desired manner. The irradiation is useful because it eliminates competition between the host organism and contaminating native organisms in grass for use of the sugars/carbohydrates produced by hydrolysis of biomass due to the cellulolytic activity of the cellulase enzymes. The irradiation method that is used in the present invention can be any radiation that is closely controllable and adjustable. The radiation source can include, but is not limited to, cobalt, cesium, and electron beam, including, but not limited to, x-ray.

More specifically, the present invention provides a method for producing cellulase enzymes for glucose and other sugar production by irradiating cellulase-producing fungi and/or bacteria or native organisms in biomass, preferably in the 1-100 kGy range, to kill most or all of the living fungi and/or bacteria while destroying minimal cellulolytic activity. The cellulase-containing fungi and/or bacteria and/or biomass is mixed with biomass to hydrolyze the biomass into sugars, including glucose, xylose, and other components. Preferably, the radiation is produced in a range of between 1 and 30 kGy.

In a preferred embodiment, a culture of fungus or bacteria is concentrated by mild centrifugation, then irradiated at a dose rate of between 6-80 kgray/minute. The dead organism mycelia can be separated from the cellulase by application of a mild shear force within a rotor-stator device such as a Supraton having slotted or tooth and chamber tools. The slurry then can enter a tank in which a charged field is imposed, and pH adjustments can be made which induce the cellulase protein to release from the mycelia. The slurry can then centrifuged aggressively to separate the cellulase protein from the mycelia. The cellulase protein can then pumped into another tank in which plastic-coated charged surfaces attract the charged cellulase protein. Once sufficient percentages of the protein have attached to the charged surfaces, the incoming slurry can be stopped and the system can be completely drained. Once drained, the cellulase is removed and recovered by cutting the current creating the charged fields on the surfaces, and an air blower can be turned on, pumping the air to a large tank, passing an air cyclone to concentrate the solids. The solids are allowed to fall in the tank and are then recovered. The enzymes can then mixed with freshly prepared biomass for hydrolysis. This procedure can be replicated a number of times until the cellulolytic activity in the enzymes has diminished to the point where they become uneconomical to re-use. In an alternate embodiment, the freshly produced, irradiated enzymes can be separated as above but without the steps describing field attraction to recover the cellulase without mycelia. In this embodiment, a portion of the mycelia can remain associated with the cellulase slurry which is mixed with freshly prepared biomass for hydrolysis.

Throughout this application, author and year, and patents, by number, reference various publications, including United States patents. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

1. A method for producing fermentable carbohydrates, plant leaf protein, and lignin, by adding cellulase enzyme complexes which are expressed from and on irradiated cellulase-complex-producing organisms, the organisms having been treated with sufficient radiation to kill biological activity after cellulase production, without destroying all cellulase enzyme complex activity, to biomass.

2. The method according to claim 1, wherein said adding step includes adding cellulase-complex enzymes expressed from and on irradiated cellulase-complex-producing organisms to dry biomass.

3. The method according to claim 1, wherein said adding step includes adding cellulase enzyme complex expressed from and on irradiated cellulase complex-producing organisms, to wetted biomass.

4. The method according to claim 1, further including the step of concentrating the partially hydrolyzed biomass and the cellulase complex mixture in water.

5. The method according to claim 4, wherein said concentrating includes allowing biomass solids to settle in the water.

6. The method according to claim 5, wherein said concentrating step includes concentrating actively hydrolyzing biomass and cellulase complex mixture, and mixing the concentrated biomass with other concentrated biomass in water.

7. The method according to claim 6, wherein said concentrating step includes mechanically concentrating the biomass.

8. The method according to claim 6, wherein said concentrating step includes adding additional irradiated cellulase complexes expressed from and on cellulase-producing organisms.

9. The method according to claim 6, wherein said concentrating step further includes irradiating the concentrated biomass and cellulase complex.

10. The method according to claim 1, further including recycling the enzyme complex.

11-14. (canceled)

15. The method according to claim 1, further including shearing and cavitating the organisms to disrupt the structure of the organisms to facilitate separation of mycelia and/or cell walls from cellulase enzyme complex, and to aid in destroying living cells.

16. The method according to claim 1, further including separating the cellulase enzyme from cellular material of the cellulase-producing organism.

17. The method according to claim 16, wherein said separating step includes separating the enzyme using methods selected from the group consisting essentially of shear and cavitation, breaking it down, and mechanically separating cells from the enzyme complex.

18. The method according to claim 1, further including pretreating the biomass for structural breakdown and increased surface area prior to adding the irradiated cellulase-producing organisms containing a cellulase complex, to the biomass.

19. The method according to claim 18, wherein said preheating step includes irradiating the biomass prior to adding the irradiated cellulase-enzyme containing organisms to the biomass.

20. The method according to claim 18, wherein said pretreating step includes shearing and cavitating the biomass prior to adding the irradiated cellulase enzyme complex-containing organisms to the biomass.

21. Irradiated cellulase-producing organisms for use in converting biomass to fermentable sugars, plant leaf protein, and lignin.

22. (canceled)

23. A method for producing cellulase enzymes for glucose and other sugar production and protein and lignin extraction by irradiating cellulase-producing organisms, thereby producing the cellulase enzymes.

24. The method according to claim 23, wherein said irradiating step includes irradiating the cellulase-producing organism with sufficient radiation to destroy biological activity without destroying all cellulase enzyme complex activity.

25. The method according to claim 24, wherein said irradiating step includes irradiating the cellulase-producing organism with radiation preferably in the range of 0.0001 kGy to 100 kGy.

26. The method according to claim 23, wherein said irradiating step includes mechanically extracting and concentrating actively-hydrolyzing biomass and a cellulase complex prior to irradiating the cellulase-producing organisms.

27-34. (canceled)

35. A method of for producing enzymes and enzyme products for by irradiating enzyme-producing organisms or cells, thereby producing the enzymes without destroying all enzyme complex activity, to a composition capable of producing enzymatic products.

36. (canceled)
